(19) United States
(12) Patent Application Publication
(10) Pub. No.: US 2024/0138715 A1
(43) Pub. Date: May 2, 2024

(54) SUPER-REGENERATIVE OSCILLATOR INTEGRATED METAMATERIAL LEAKY WAVE ANTENNA FOR MULTI-TARGET MOTION DETECTION AND RANGING

(71) Applicant: Rutgers, The State University of New Jersey, New Brunswick, NJ (US)

(72) Inventors: Chung-Tse Michael Wu, New Brunswick, NJ (US); Yichao Yuan, New Brunswick, NJ (US)

(73) Assignee: Rutgers, The State University of New Jersey, New Brunswick, NJ (US)

(21) Appl. No.: 18/384,056

(22) Filed: Oct. 26, 2023

Related U.S. Application Data
(60) Provisional application No. 63/419,510, filed on Oct. 26, 2022.

(51) Int. Cl. A61B 5/11; A61B 5/05; G01S 7/03

(52) U.S. Cl. CPC A61B 5/1126; A61B 5/05; A61B 5/1102; G01S 7/03

(57) ABSTRACT

Various embodiments comprise systems, methods, architectures, mechanisms and apparatus providing a super-regenerative oscillator (SRO) integrated metamaterial (MTM) leaky wave antenna (LWA) architecture suitable for use in radio frequency (RF) detecting, ranging (e.g., RADAR), and sensing systems/applications, such as a noncontact multi-target vital sign detection system.

### CROSS-REFERENCE TO RELATED APPLICATION

This application claims priority to U.S. Provisional Patent Application No. 63/419,510, filed on Oct. 26, 2022, entitled SUPER-REGENERATIVE OSCILLATOR INTEGRATED METAMATERIAL LEAKY WAVE ANTENNA FOR MULTI-TARGET MOTION DETECTION AND RANGING (Attorney Docket No. RU-2022-049), which application is incorporated herein by reference in its entirety.

### GOVERNMENT INTEREST

This invention was made with government support under grant number 1818478 awarded by the NSF. The government has certain rights in the invention.

### FIELD OF THE DISCLOSURE

The present disclosure generally relates to radar sensing and, in particular, to multi-target vital sign and motion detection.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Real-time cardiovascular disease (CVD) monitoring in daily life is one of the effective methods to prevent CVDs. During the past decade, human vital sign signal detection, such as respiration rates (RRs) and heartbeat rates (HRs), based on wearable and noncontact methods attracts considerable attention. Most of the existing monitoring methods are based on wearable solutions, such as finger pulse meters, smart watches, or electrocardiograms (ECGs).

The cardiac activity of human target causes heart shrinking and swelling, as well as the volume of chest increasing and decreasing periodically. These periodic movements can be detected using Doppler radar sensor, which has been one of the promising noncontact methods for vital sign detection. In the Doppler radar system, a radiofrequency (RF) signal is transmitted by the transmitter (Tx) toward the detected target, modulated by the cardiac movement and then reflected from the target. Then the vital sign information can be extracted from the down-converted received RF signal. To this end, several sensing techniques, such as continuous wave (CW) and frequency modulated continuous wave (FMCW), have been employed for medical applications in radar systems.

Although radar sensors can be used to perform vital sign monitoring in a noncontact manner, only single target can usually be detected using omnidirectional antennas. Several radar architectures for multi-target vital sign detection have been studied, including adopting mechanical rotors, multiple-input multiple-output (MIMO) or phased array. However, mechanical devices can be used to control the radiation direction of radar system directly, while they are bulky and slow in response. Although multiple targets can also be distinguished by MIMO and phased arrays, which are based on multiple antennas cooperating with each other, they are more costly and complex owing to additional control schemes and RF chains, which may limit their applications.

In addition to multi-target vital sign detection, there are still some limitations for vital sign detection using radar sensors in the daily life. A great challenge is how to provide the high detection accuracy due to the small cardiac movement and sensitivity limitation from the system. Also, the low power consumption and reduced circuit complexity are much desired since they can lead to smaller system size and longer battery life. As compared with vital sign detection based on conventional homodyne radar architecture, a simpler hardware system with higher detection sensitivity and accuracy can be achieved in the so-called self-injection-locked (SIL) radar architecture proposed recently. However, the SIL-based radar sensor still suffers the null point issue in every quarter wavelength from the radar to the target. To overcome this issue, quadrature coupler or additional complex techniques, such as frequency-tuning technique, can be used at the expense of a larger size and higher system complexity. Besides, sufficient amount of received power back-scattered from the target is required in the SIL-based radar to achieve the self-injection-locked state.

### SUMMARY

Various deficiencies in the prior art are addressed by systems, methods, architectures, mechanisms and apparatus providing a super-regenerative oscillator (SRO) integrated metamaterial (MTM) leaky wave antenna (LWA) architecture suitable for use in radio frequency (RF) detecting, ranging (e.g., RADAR), and sensing systems/applications, such as a motion sensing and noncontact multi-target vital sign detection system.

A radio frequency (RF) motion sensor according to one embodiment comprises: a metamaterial (MTM) leaky wave antenna (LWA) configured to transmit an interrogating RF signal toward each of a plurality of targets and to correspondingly receive therefrom respective target reflected signal; and a super-regenerative oscillator (SRO) comprising a tunable voltage-controlled oscillator (VCO) configured to generate for transmission by the MTM LWA an oscillating driver signal in accordance with a sequence of quench cycles, wherein an amplitude of the oscillating signal changes from an initial value to an equilibrium value (V_{eq}) and back to an extinction value during a first portion of each quench cycle; and a demodulator configured to demodulate from the MTM LWA received signals to provide thereby respective baseband signals reflected from different targets, which includes the target-associated motion information.

Additional objects, advantages, and novel features of the invention will be set forth in part in the description which follows and will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 depicts an embodiment of a super-regenerative oscillator (SRO)-integrated radar sensor structure;

FIGS. 2A and 2B depict embodiments of SRO-integrated metamaterial (MTM) leaky wave antenna (LWA) sensors suitable for use in a multi-target vital sign detection system;

FIG. 3A depicts a layout of a 1D MTM LWA with MTM-based coupler according to an embodiment;

FIG. 3B graphically illustrates a radiation pattern of the 1D MTM LWA of FIG. 3A;

FIG. 4A depicts a layout of a voltage-controlled oscillator (VCO) under SRO mode according to an embodiment;

FIG. 4B graphically illustrates oscillation frequency with respect to bias voltage (Vₜᵤₙₑ) of a varactor within the VCO of FIG. 4A;

FIG. 5 graphically illustrates the damping factor, received signal, and output signal of the SRO-integrated radar sensor in the logarithmic mode;

FIG. 6 depicts an image of a SRO-integrated MTM LWA sensor;

FIG. 7 graphically illustrates time-domain waveforms of a quench signal and a down-converted output signal of a SRO-integrated MTM LWA sensor;

FIG. 8 graphically depicts a top view sketch of a measurement experiment setup for vital sign detection of multi-site targets;

FIG. 9A graphically illustrates normalized spectrum of measured respiration rate (RR) and heartbeat rate (HR) for a first target at different distances under different modes;

FIG. 9B graphically illustrates normalized spectrum of measured RR and HR for a second target at different distances under different modes.

FIG. 10 graphically depicts a top view sketch of a measurement experiment setup for actuator motion detection;

FIG. 11 graphically illustrates measured received spectrum of a 1 Hz actuator motion detection experiment at different displacements;

FIG. 12 graphically illustrates measured received spectrum of the 2.5 Hz actuator motion at different distances under different modes;

FIG. 13 depicts a high-level block diagram of a system according to various embodiments;

FIG. 14 depicts a block diagram of SRO-integrated metamaterial (MTM) leaky wave antenna (LWA) radar system for multi-target vital sign detection and location tracking in accordance with various embodiments;

FIG. 15A graphically illustrates measured oscillation frequency as a function of varactor bias voltage of a 1D MTM LWA in the system of FIG. 14 according to an embodiment;

FIG. 15B graphically illustrates a radiation pattern of a 1D MTM LWA in the system of FIG. 14 according to an embodiment;

FIG. 16A graphically illustrates a measured time-domain waveform of an output signal from a MTM-based coupler (down converted to 3 MHz and having a quench signal at 100 Hz);

FIG. 16B graphically illustrates a comparison of oscillation time width between different quench cycles (without an envelope detector);

FIG. 17 graphically depicts distance measurement using a cross-correlation method in the time domain according to an embodiment;

FIG. 18A graphically illustrates measured distance results associated with an experiment using an actuator at distances of 79 cm and 62 cm;

FIG. 18B graphically received spectrum associated with the experiment using an actuator at 62 cm;

FIG. 19 depicts an experiment setup and top-view sketch of a SRO-based MTM pulsed radar for multi-target vital sign detection and location tracking;

FIG. 20A graphically illustrates measured breathing rate and heartbeat for a first target in the experiment of FIG. 19; and

FIG. 20B graphically illustrates measured breathing rate and heartbeat for a second target in the experiment of FIG. 19.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments may be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Various embodiments provide systems, methods, architectures, mechanisms and apparatus providing a metamaterial (MTM) leaky wave antenna (LWA) integrated with a super-regenerative oscillator (SRO) architecture and suitable for use in radio frequency (RF) detecting, ranging (e.g., RADAR), and sensing systems/applications, such as a noncontact multi-target vital sign detection system. As will be described in more detail below, the various embodiments include a metamaterial (MTM) leaky wave antenna (LWA) based on a super-regenerative oscillator (SRO) architecture that can be used to detect multi-target vital sign information and motion, without complex systems or contacting targets. Vital sign information for multi-site targets is detected accurately at a farther distance, compared with radar sensors based on self-injection-locked (SIL) architecture. One biological application is vital sign or motion detection for multiple targets at different locations. The disclosed SRO-integrated MTM LWA can achieve higher sensitivity, lower power consumption, as well as excellent null point and steady cluster immunity with reduced system complexity for multi-target vital sign detection. The disclosed SRO-integrated MTM LWA can also be used in a portable device for different application scenarios, such as life searching under earthquake ruins, due to its low power consumption.

In one embodiment, a proposed radar sensor comprises a one-dimensional (1D) MTM LWA and a tunable voltage-controlled oscillator (VCO), the sensor being configured to perform frequency-dependent space mapping with the main-beam angle sweeping from −50° to +30° with respect to the varying, frequency from 1.85 to 2.85 GHz.

In other embodiments, the proposed radar sensor comprises a two-dimensional (2D) MTM LAVA and a tunable voltage-controlled oscillator (VCO), the sensor being configured to perform frequency-dependent space mapping.

The disclosed SRO-integrated MTM LWA system may be operated in a logarithmic mode with a quench signal imposed at the drain port of transistor. Experiments have been conducted to show that vital sign information for two human targets is successfully detected when they are located along different scanning angles of −10° and −30°, respectively. Furthermore, compared with MTM LWA radar sensor based on the self-injection-locked (SIL) architecture, vibrating motion at a farther distance is detected accurately using the disclosed radar sensor, indicating a higher sensitivity with reduced system complexity.

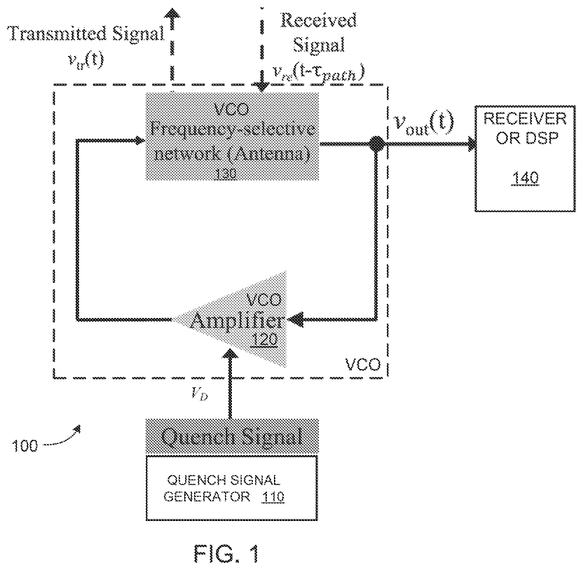

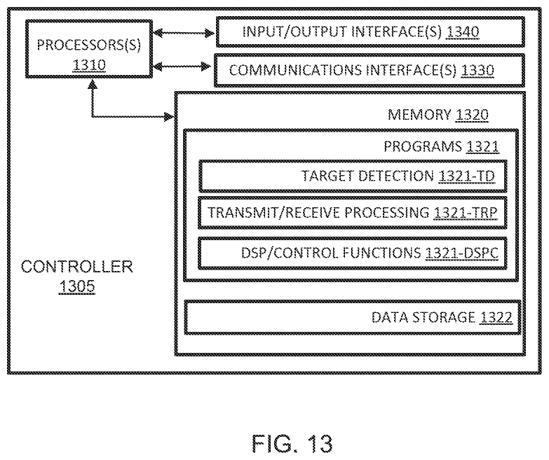

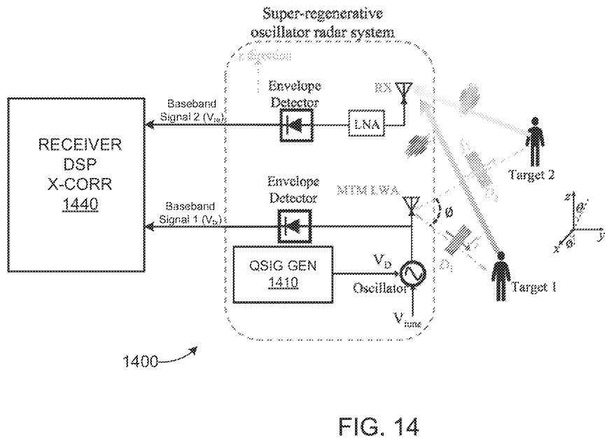

FIG. 1 depicts a simplified SRO-integrated sensor structure. Specifically, in the SRO-integrated sensor structure 100 of FIG. 1, a tunable voltage-controlled oscillator (VCO) is formed using an amplifier 120 and frequency-selective network 130, wherein the amplifier 120 drives the frequency-selective antenna 130 to cause the antenna to transmit a radio frequency (RF) signal vₜᵣ (t) towards at least one target and to receive from the each target a respective reflected signal vᵣₑ(t−τₚₐₜₕ), which is detected and demodulated to produce an output signal vₒᵤₜ (t), which is fed back to the amplifier 120 and also subjected to further processing to extract therefrom the target information such as via a signal receiver or digital signal processor (DSP) 140, which may comprise a signal processing system having a built in analog to digital converter (ADC) or other suitable receiving/processing device. The signal receiver 140 and quench signal generator 110 may be implemented in a standard manner such as by using off-the-shelf commercial equipment/components as will be appreciated by those skilled in the art, such as described below with respect to FIGS. 13-14.

A quench signal v_{D} is imposed at the amplifier 120 in a feedback loop to control thereby the gain of the amplifier 120, which makes the sensor output cyclically switch between an oscillation occurrence state and an oscillation extinction state. That is, the amplitude of the oscillating signal changes from an initial value to an equilibrium value (V_{eq}) resulting in an oscillation state, and back to an extinction value resulting in an extinction state, during a first portion of each quench cycle.

During an intrinsic “sensitivity period” of each quench cycle, the SRO-integrated radar sensor 100 is susceptible to the received signal. A tiny signal disturbance in the received signal has a large influence on the oscillation state. Therefore, the SRO-integrated radar sensor exhibits higher sensitivity than that from a SIL-based radar sensor architecture. It is known by the inventors that a 34-39 dB relative voltage gain improvement for peak value of the output spectrum in the SRO-integrated radar sensor can be achieved compared with SIL-based radar sensor architecture. Furthermore, the SRO-integrated radar sensor has the advantage of null-point and dc-offset immunity, which is usually caused by steady clusters in the detected environment, because the oscillation build-up time within each quench cycle is influenced by the received signal with amplitude rather than phase modulation. Consequently, compared to the SIL-based radar sensor, a smaller system size with lower cost can be achieved in the SRO-integrated radar sensor architecture due to the removal of quadrature coupler.

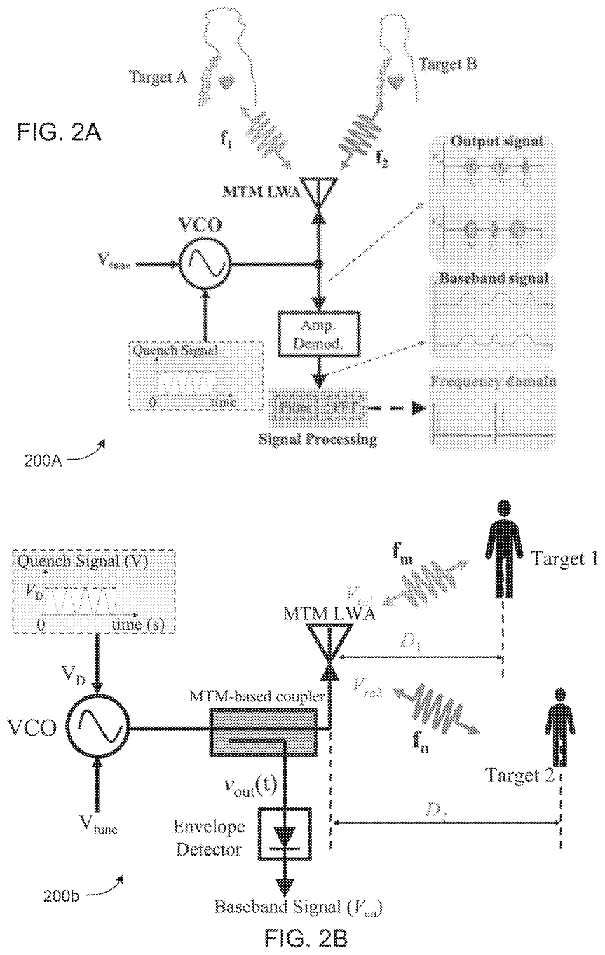

FIGS. 2A and 2B depict embodiments of SRO-integrated metamaterial (MTM) leaky wave antenna (LWA) sensors suitable for use in a multi-target vital sign detection system. Specifically, the SRO-integrated MTM LWA sensors 200A/200B of FIGS. 2A/2B comprise noncontact SRO-integrated MTM LWA sensors suitable for use in multi-target vital sign detection and other applications.

As depicted in FIG. 2A, a voltage-controlled oscillator (VCO) having an amplified output signal of a frequency selected in accordance with a DC input tuning voltage (Vₜᵤₙₑ) drives a MTM LWA, causing the MTM LWA to transmit, illustratively, two radio frequency (RF) signals f₁ and f₂ toward respective targets A and B, and to receive reflected signals therefrom. The reflected signals are detected and demodulated to provide corresponding baseband reflected signals, which baseband reflected signals are subjected to signal processing (e.g., filtering, fast Fourier transform (FFT), etc.) to provide thereby frequency domain information pertaining to the targets A and B, such as motion indicative of respiration rate (RR) and the like.

A quench signal v_{D} is imposed at the VCO amplifier to control thereby the gain of the amplifier, which makes the sensor output cyclically switch between an oscillation occurrence state and an oscillation extinction state. During an intrinsic “sensitivity period” of each quench cycle, the SRO-integrated MTM LWA sensor is susceptible to the received (reflected) signals from the targets A and B.

Since the oscillation amplitude within each quench cycle is modulated by, in this example, cardiac activity of the targets A and B, an envelope detector implemented for baseband signal processing is adapted for vital sign information extraction from the received signals reflected from the targets A and B.

Similar to the SRO-integrated sensor structure 100 of FIG. 1, which uses a quench signal to control amplifier gain and force the system into occurrence and extinction states, the SRO-integrated MTM LWA sensors of FIGS. 2A/2B are configured to use a VCO to realize a super-regenerative circuit operating in a logarithmic mode rather than stable state mode. Specifically, the SRO-integrated MTM LWA sensors of FIGS. 2A and 2B depict are operated in a logarithmic mode in which sensitivity is enhanced during an intrinsic sensitivity period of the SRO such that these sensors not only exhibit multi-target detection capability, but also have higher sensitivity using a more straightforward architecture and without requiring additional hardware devices or complex algorithms. In the logarithmic mode, the amplitude of the oscillation always rises up and reaches to the equilibrium value (V_{eq}) within each quench cycle, while the build-up area under the envelope is modulated by the received signal reflected from the target.

As shown in FIG. 2B, the MTM LWA sensor 200b possesses dual functions; namely, it transmits the interrogating RF signals toward one or more targets as well as receives the reflected signal(s) from the target(s). The operating frequency of the sensor system is provided by a tunable voltage-controlled oscillator (VCO) that may be used to control the illuminating direction of the MTM LWA. To enable the sensor system working under the SRO mode, a sinusoidal waveform is utilized to serve as the quench signal in the oscillator circuit. The reflected signal influenced by the physiological activity of human target, such as heart shrinking and swelling, is sent into an envelope detector through an MTM-based coupler for amplitude demodulation.

In this scenario, the main beam direction of MTM LWA, θ(ω), can be expressed as:

$$θ ( ω ) = arc sin ( \frac{β ( ω )}{{k}_{0}} ) \qquad ( 1 )$$

where k₀ is the free space wavenumber, and the propagation constant of CRLH LWA β(ω) is a function of the frequency which can be changed from negative to positive value as the frequency increases.

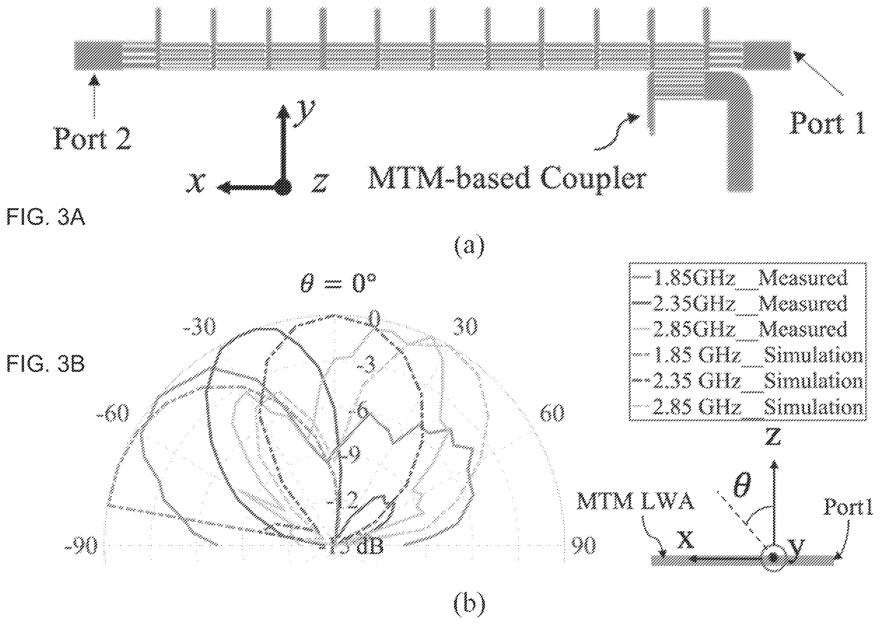

FIG. 3A depicts a layout of a 1D MTM LWA with MTM-based coupler according to an embodiment, and FIG. 3B graphically illustrates a radiation pattern of the 1D MTM LWA of FIG. 3A.

To experimentally verify the frequency-dependent space mapping behavior of MTM LWA and direct the RF signal into the following circuit for data postprocessing, a one-dimensional (1D) MTM LWA is integrated with an MTM LWA coupler, in which the operating frequency covers the 2.4 GHz band, as shown in FIG. 3(a). As can be seen from the simulated and measurement results in FIG. 3(b), the scanning angle range for the main beam angle is from −50° to +30° with respect to the varying frequency from 1.85 to 2.85 GHz. A small deviation between the simulation and measurement is mainly caused by the in-house fabrication error.

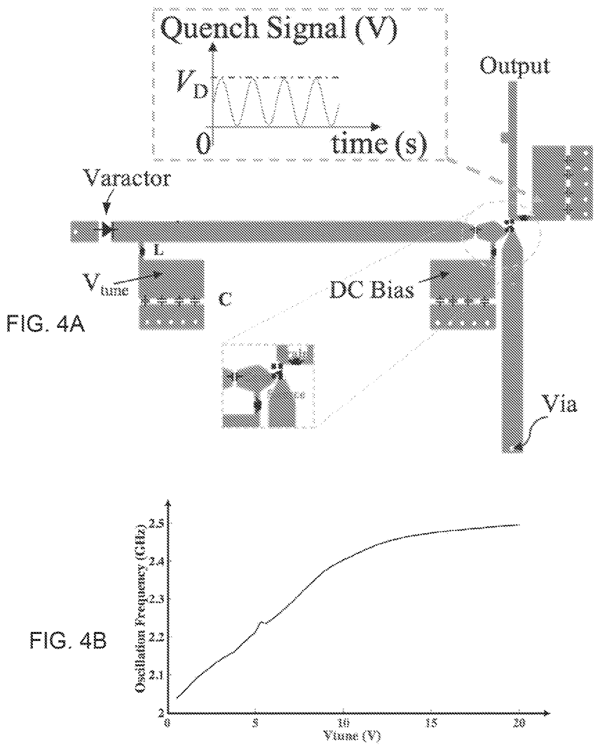

FIG. 4A depicts a layout of a voltage-controlled oscillator (VCO) under SRO mode according to an embodiment, and FIG. 4B graphically illustrates oscillation frequency with respect to bias voltage of a varactor (Vₜᵤₙₑ) within the VCO of FIG. 4A.

As shown in FIG. 4A, a VCO circuit with common source configuration is designed based on negative resistance method. As expected, the corresponding oscillation frequency adjustment range covers the band from 2.04 GHz to 2.48 GHz with respect to the changing bias voltage of varactor (Vₜᵤₙₑ) from 0 to 20 V, as shown in FIG. 4(b). The instantaneous damping factor ζ(t) of the entire sensor system can be expressed as:

ζ(t)=ζ₀(1−AₘₐₓAₐ(t)) (2)
where ζ₀ is the quiescent damping factor, Aₘₐₓ is the maximum amplification of the frequency-selective network, shown in FIG. 1. Aₐ(t) is the gain of the transistor controlled by the quench signal (V_{D}) through the drain of the transistor.

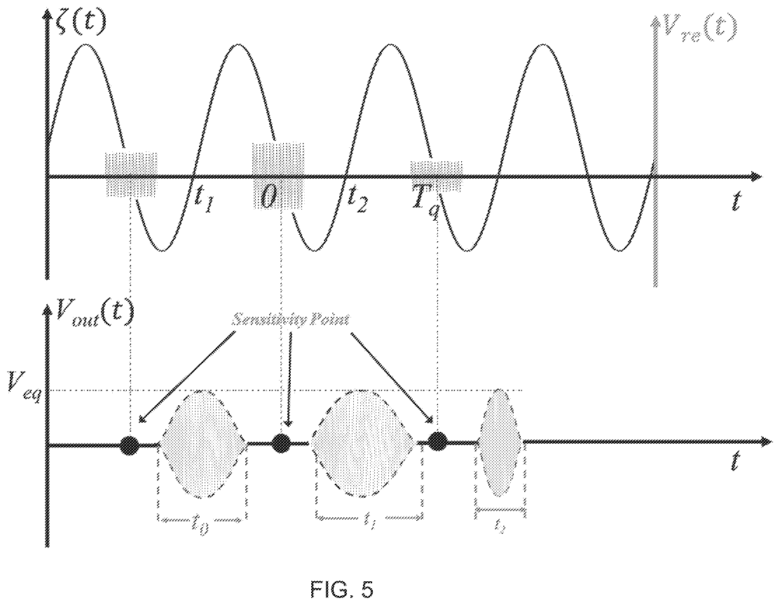

FIG. 5 graphically illustrates time domain waveforms of the SRO radar. Specifically, FIG. 5 depicts damping factor (ζ(t)), received signal (Vᵣₑ), and output signal (Vₒᵤₜ) of the SRO-integrated radar sensor in a logarithmic mode of operation.

To eliminate the influence from the amplitude change of transmitted signals on the amplitude modulation due to cardiac activity, the proposed SRO-integrated radar sensor is designed to operate in the logarithmic mode. In the logarithmic mode, the amplitude of the oscillation always rises up and reaches to the equilibrium value (V_{eq}) within each quench cycle, while the build-up area under the envelope is modulated by the received signal reflected from the target. As shown in FIG. 5, for one quench cycle, e.g., at t=0 when ζ(t) crosses zero, the SRO-integrated radar sensor is susceptible to tiny, received disturbance, which causes the generation and rise of the oscillation for the sensor system. After reaching to the equilibrium value (V_{eq}) at t=t₂, the oscillation phenomenon starts decaying and dies out before the quench period ends at t=T_{q}.

Assuming the transmitted power from the SRO-integrated radar sensor is Pₜᵣ within one quench period in the logarithmic mode, based on the two-way radar equation, the received power (Pᵣₑ) for target k (k=1, 2 in FIG. 2) is:

$$\frac{{P}_{rek}}{{P}_{tr}} = {G}_{a n t k}^{2} \frac{{λ}_{k}^{2} {σ}_{k}}{{( 4 π )}^{3} {D}_{k}^{4}} \qquad ( 3 )$$

where λₖ is the wavelength depending on the operating frequency which corresponds to target k's direction, Gₐₙₜₖ and σₖ are the maximum antenna gain and the radar cross section with respect to the detected target, respectively, and DK is the distance to the detected target, as shown in FIG. 2.

Thus, the power change caused by the small displacement (Δd) of the detected target is given by:

$$\frac{{P}_{rek ( {D}_{k} + Δ {d}_{k} )}}{{P}_{rek ( {D}_{k} )}} = \frac{{D}_{k}^{4}}{{( {D}_{k} + Δ {d}_{k} )}^{4}} \qquad ( 4 )$$

which leads to a voltage ratio of:

$$\frac{{V}_{rek ( {D}_{k} + Δ {d}_{k} )}}{{V}_{rek ( {D}_{k} )}} = \frac{{D}_{k}^{2}}{{( {D}_{k} + Δ {d}_{k} )}^{2}} . \qquad ( 5 )$$

In the logarithmic mode, the amplitude change during the oscillation build-up procedure can be expressed as an exponential function:

$${V}_{e q k} = {V}_{rek} {e}^{\frac{{t}_{k}}{{τ}_{0 k}}} \qquad ( 6 )$$

where V_{eqk} is the equilibrium value of the output signal and τ₀ₖ is the time constant for the oscillation build-up. The average value of the output signal from the envelope detector can be expressed as:

$$\overline{{V}_{e n k}} = \overline{{V}_{refk}} + {f}_{q} {τ}_{0 k} {V}_{e q k} ln ( \frac{{V}_{rek}}{{V}_{refk}} ) \qquad ( 7 )$$

where V̅_̅{̅r̅e̅f̅k̅}̅ and V_{refk} are the average value and the initial value of the reference amplitude of output signal for target k, respectively, and f_{q} is the quench frequency.

Based on equations (5) and (7), the variation of the average output signal from the envelope detector for different quench cycles is related to the small displacement as follows:

$$Δ \overline{{V}_{e n k}} = \overline{{V}_{e n k ( {D}_{k} )}} - \overline{{V}_{e n k ( {D}_{k} + Δ {d}_{k} )}} = \frac{2 {f}_{q} {τ}_{0 k} {V}_{e q k}}{{D}_{k}} Δ {d}_{k} . \qquad ( 8 )$$

Moreover, the build-up time difference for different quench cycles can be obtained as:

$$Δ {t}_{k} = {t}_{k ( {D}_{k} + Δ {d}_{k} )} - {t}_{k ( {D}_{k} )} = {τ}_{0 k} ln ( \frac{{V}_{rek ( {D}_{k} )}}{{V}_{rek ( {D}_{k} + Δ {d}_{k} )}} ) = \frac{2 {τ}_{0 k} Δ {d}_{k}}{{D}_{k}} . \qquad ( 9 )$$

As can be seen in equation (9), the build-up time difference for the two different quench cycles is proportional to the small cardiac movement of the target (Δ_{dk}). With the movement amplitude increased, the build-up time difference between different quench cycles will be increased, which contributes to a larger amplitude in the spectral domain.

In so doing, the fast Fourier Transform (FFT) is then performed on the baseband signal after conducting the demodulation by the envelope detector to extract the vital sign information.

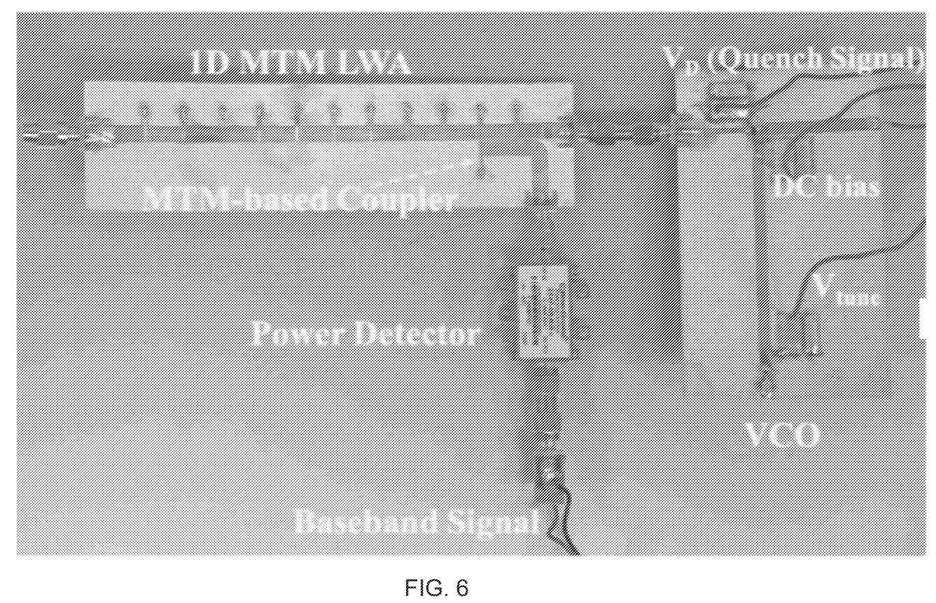

FIG. 6 depicts an image of an SRO-integrated MTM LWA sensor. Specifically, FIG. 6 depicts a prototype of the disclosed SRO-integrated MTM LWA. The MTM LWA and VCO are implemented on a Rogers RT/duroid 5880 LZ substrate with a thickness of 1.27 mm, a dielectric constant of 2 and loss tangent (tan δ) of 0.0027. In this case, detection is made along a specified direction with the interrogating RF signal transmitted from the 1D MTM LWA based on its radiation pattern. The reflected signal containing the vital sign information of human target is received by the same MTM LWA and coupled to a commercial power detector, ZL47-40LN-S+, for amplitude demodulation. Then, the baseband signal is obtained by the Tektronix MDO 34 oscilloscope with a sampling frequency of 500 KHz. The quench signal, which may be provided by a function generator or oscillator, as used herein comprises is a sinusoidal signal biased at 1.1 V with an amplitude of 1.1 V at 100 Hz, which leads to the logarithmic mode for the entire SRO radar/sensor system. Modifications to the type and shape and properties of the quench signal are also contemplated to be useful within the context of the various embodiments (differing amplitudes, shapes, frequencies, etc.).

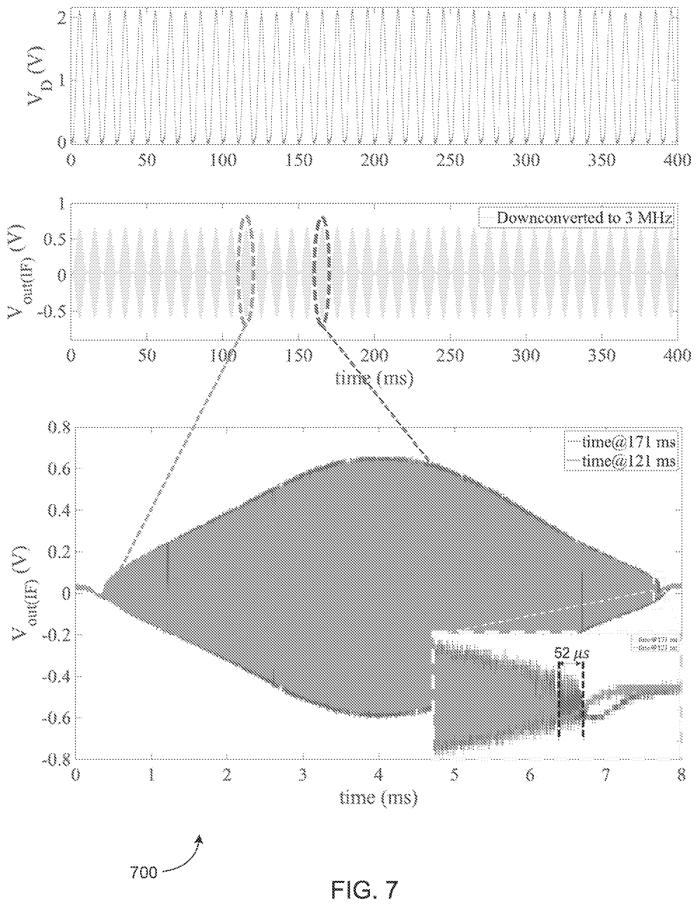

FIG. 7 graphically illustrates the measured time-domain waveform of the quench signal at the drain of the transistor and output signal of the SRO-integrated MTM LWA sensor (down converted to 3 MHz) with oscillation time width comparison.

FIG. 7 shows the measured quench signal (V_{D}) and the down-converted output signal (Vₒᵤₜ(In) at 3 MHz, along with the oscillation time width comparison for two different quench cycles resulting from a small displacement generated by small hand movements. A time difference of 52 μs with similar peak amplitude can be observed, which indicates the logarithmic mode operation for the proposed SRO-integrated MTM LWA and coincides with the waveform of FIG. 5.

To evaluate the performance of the designed circuit, the radar sensor is first arranged to detect vital sign information for multiple targets at different locations. To minimize the interference between the two targets, two human targets along the direction of 0=−30° and 0=−10° at different distances are chosen, where their angular difference is larger than the −3 dB beamwidth of the main-beam (20° in this case) according to the radiation pattern shown in FIG. 3(b). Two corresponding oscillation frequencies of 2.24 GHz and 2.43 GHz can be used based on the radiation pattern shown in FIG. 3(b), respectively.

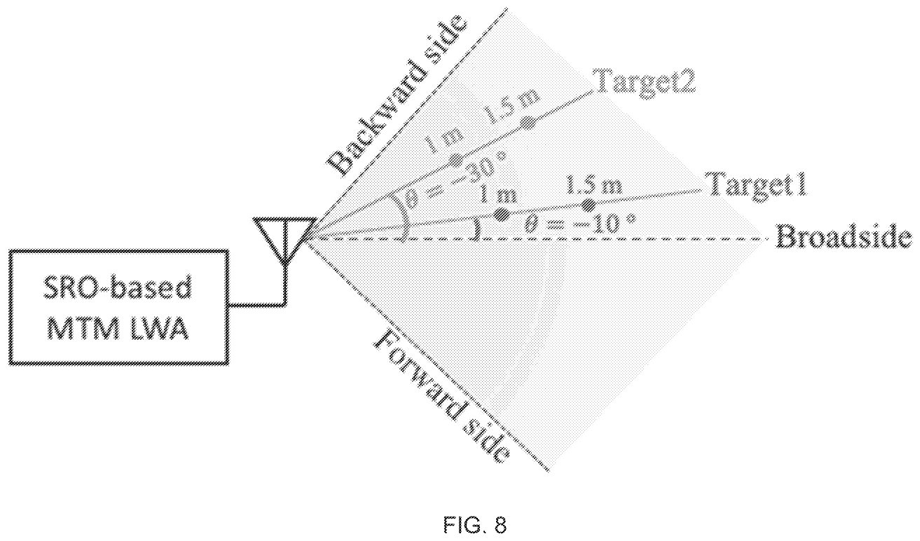

FIG. 8 graphically depicts a top view sketch of a measurement experiment setup for vital sign detection of multi-site targets (e.g., two targets). Respiration rate (RR) and heartbeat rate (HR) for both targets can be measured from the frequency spectrum through a fast Fourier transform (FFT) method. Furthermore, the disclosed radar sensor can be operated under the SIL mode when a constant drain voltage of 2 V is imposed. For converting the RF output signals from the radar sensor into baseband signals, an additional first-order microwave differentiator is incorporated for Frequency Modulation (FM) to Amplitude Modulation (AM) conversion. Meanwhile, a quadrature coupler with envelope detectors for complex demodulation is adopted.

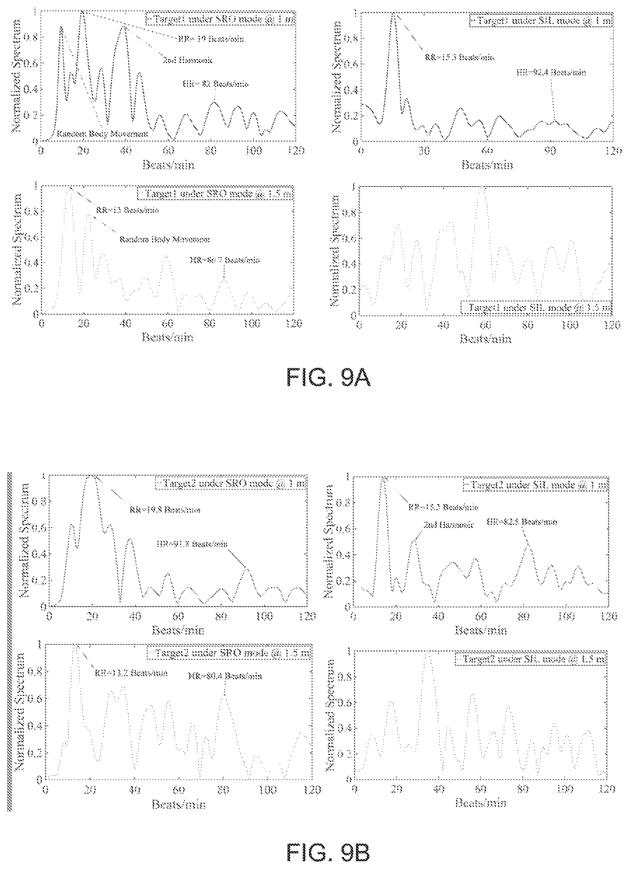

FIG. 9A graphically illustrates normalized spectrum of measured respiration rate (RR) and heartbeat rate (HR) for a first target at different distances under different modes, while FIG. 9B graphically illustrates normalized spectrum of measured RR and HR for a second target at different distances under different modes. In particular, FIGS. 9A-9B show the measured RRs and HRs for both targets at different distances under the SRO and SIL modes when a Hanning window and a high pass filter with a cut off frequency of 0.2 Hz are implemented. The RRs and HRs are obtained from different frequency ranges of 0-40 beats/min and 50-120 beats/min, respectively. At the distance of 1 m, the RRs for Target 1 under SRO and SIL modes are 19 beats/min and 15.3 beats/min, while the ground truth are 18 beats/min and 16.5 beats/min, respectively. The HRs for Target 1 under SRO and SIL modes are 82 beats/min and 92.4 beats/min, while the ground truth are 85 beats/min and 91 beats/min, respectively. Some low frequency spikes can be seen in the frequency domain, which may come from random body movements (RBM). Meanwhile, because of higher sensitivity for the SRO mode, the baseband signal in the SRO mode appears noisier when compared to the SIL mode. Exemplary RRs and HRs for two targets at different distances under different modes are summarized below in Tables I and II. It can be seen that the measured RRs and HRs for both targets at distance of 1 m coincide with the ground truth under SRO and SIL modes. Nevertheless, in the case of 1.5 m distance, vital sign information under the SIL mode is buried in the environment noises, which leads to the inaccurate measurement results. On the other hand, RRs and HRs can still be obtained under the SRO mode, indicating a higher sensitivity compared with the SIL mode.

Generally speaking, the oscillation frequency band of the VCO circuit does not have any preferred frequency ranges. Its frequency band may depend, for example, on the operation frequency range of the antenna(s) used.

TABLE I
VITAL SIGN DETECTION FOR TARGET1 AT DIFFERENT
DISTANCES UNDER DIFFERENT MODES

| | SRO mode | | SIL mode | |
|---|---|---|---|---|
| Distance (m) | 1 | 1.5 | 1 | 1.5 |
| Respiration Rate (Beats/min) | 19 | 13 | 15.3 | unreadable |
| Ground Truth | 18 | 16.5 | 16.5 | 19.5 |
| Heartbeat Rate (Beats/min) | 82 | 86.7 | 92.4 | unreadable |
| Ground Truth | 85 | 90 | 91 | 84 |

TABLE II
VITAL SIGN DETECTION FOR TARGET2 AT DIFFERENT
DISTANCES UNDER DIFFERENT MODES

| | SRO mode | | SIL mode | |
|---|---|---|---|---|
| Distance (m) | 1 | 1.5 | 1 | 1.5 |
| Respiration Rate (Beats/min) | 19.8 | 13.2 | 15.3 | unreadable |
| Ground Truth | 18 | 16.5 | 16.5 | 18 |
| Heartbeat Rate (Beats/min) | 91.8 | 80.4 | 82.5 | unreadable |
| Ground Truth | 92 | 83 | 89 | 91 |

TABLE III
COMPARISON OF ACTUATOR MOTION DETECTION UNDER
DIFFERENT MODES AT DIFFERENT DISTANCES

| | SRO-integrated | SIL-based |
|---|---|---|
| | MTM LWA | MTM LWA |
| d = 0.9 m | 2.5 | 2.24 |
| Absolute Error | 0 | 0.26 |
| d = 1.5 m | 2.87 | 2.5 |
| Absolute Error | 0.37 | 0 |
| d = 2 m | 2.63 | 1.38 |
| Absolute Error | 0.13 | 1.12 |

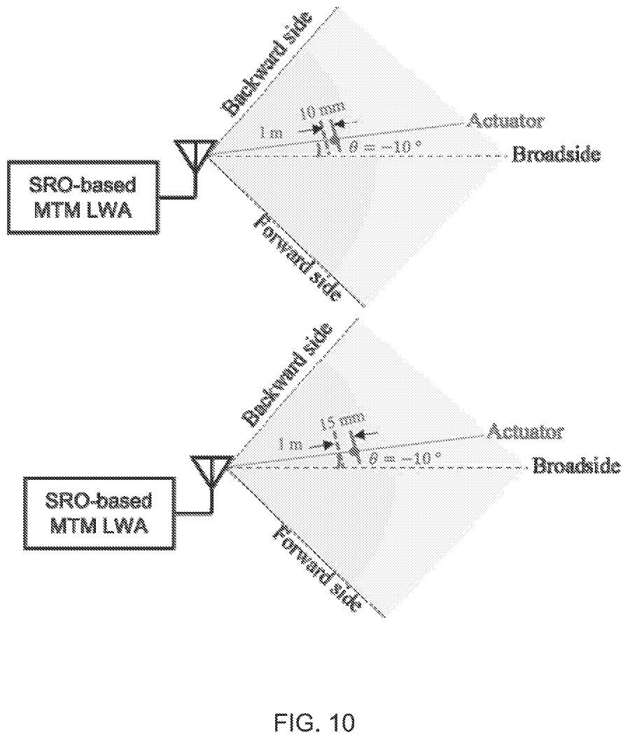

FIG. 10 graphically depicts a top view sketch of a measurement experiment setup for actuator motion detection. Specifically, to further demonstrate the high sensitivity of the disclosed SRO-integrated MTM LWA sensor, an actuator is first employed as the target placed along the direction of θ=−10° with a vibration frequency of 1 Hz.

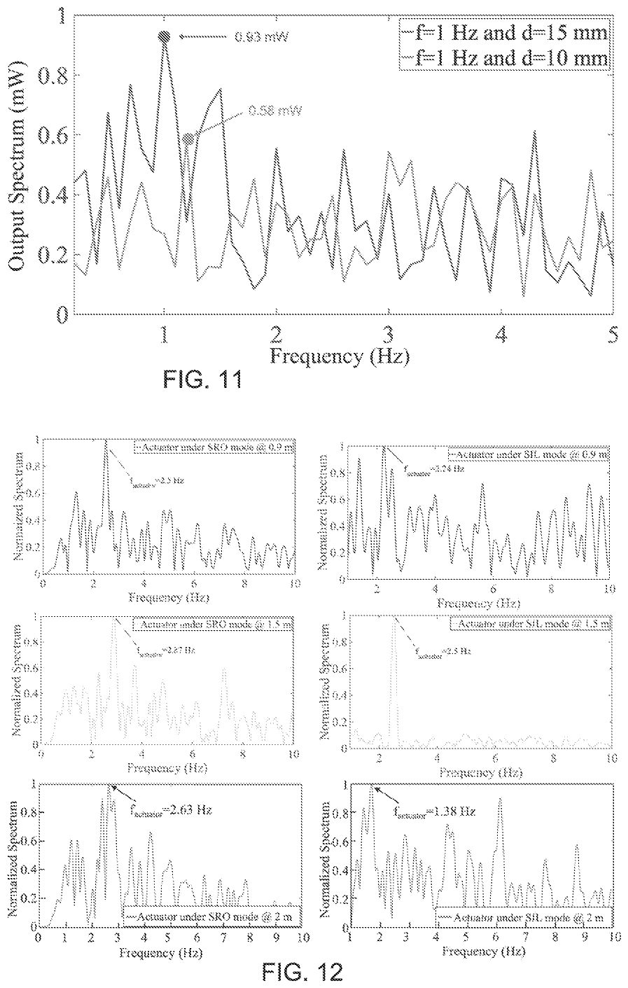

FIG. 11 graphically illustrates measured received spectrum of a 1 Hz actuator motion detection experiment at different displacements. That is, when the displacement of the actuator of FIG. 10 is changed from 10 mm to 15 mm at a distance of 1m, the measured received spectrum is obtained by performing FFT on the raw data to provide the graphical illustration of FIG. 11. The peak amplitude of frequency spectrum at the vibration frequency is changed from 0.58 mW to 0.93 mW, which coincides with the various analysis described herein, such as described above with respect to Equations (8) and (9).

FIG. 12 graphically illustrates measured received spectrum of the 2.5 Hz actuator motion at different distances, in particular, when the actuator is placed along the same direction but at different distances with the displacement of 5 mm and the vibration frequency of 2.5 Hz. By adopting the Hanning window and the high pass filter with a cut off frequency of 0.5 Hz, the measured received spectrum of the actuator at different distances are plotted in FIG. 12. It can be observed it is less noisy compared with the results in FIG. 9 because the actuator movement does not entail the RBM issues. It is worth mentioning that actuator motion can still be detected at d=1.5 m under SIL mode due to the larger doppler motion of the actuator compared with the cardiac activity of human target. Table III compares the detected frequency of the actuator motion under different modes at different distances. It can be seen there is a large absolute error for a SIL-based MTM LWA sensor at the distance of 2 m. The experiment results show that the target motion at a farther distance can be accurately detected under the SRO mode, which signifies the high sensitivity of SRO-integrated MTM LWA sensor.

The above-disclosed embodiments comprise a new kind of radar sensor that integrates MTM LWA and SRO architecture and is suitable for use in various applications, such as to detect single or multiple target vital sign signals, motion sensing, and so on. The frequency-dependent space mapping can be achieved with the implementation of a MTM LWA sensor with a tunable VCO circuit. The periodical quench signal is imposed at the drain of transistor in the oscillator circuit for the logarithmic operation mode of the radar/sensor system. The amplitude of RF output signals from MTM LWA within each quench cycle are demodulated through an envelope detector. Benefiting from the intrinsic “sensitivity period” and amplitude modulation fashion, the proposed SRO-integrated radar sensor has a great potential in increasing the detection sensitivity with reduced system complexity. Experimental results reveal that the vital sign information of multi-site targets can be detected accurately by using the proposed SRO-integrated radar sensor. Moreover, motion detection experiment signifies the proposed SRO-integrated radar sensor exhibits a higher accuracy with less absolute error value over longer distances compared with the SIL-based MTM LWA sensor.

A method of sensing motion in accordance with the various embodiments may comprise, illustratively, generating, using a super-regenerative oscillator (SRO) comprising a tunable voltage-controlled oscillator (VCO), an oscillating driver signal in accordance with a sequence of quench cycles, wherein an amplitude of the oscillating signal changes from an initial value to an equilibrium value (V_{eq}) and back to an extinction value during a first portion of each quench cycle; transmitting, via a metamaterial (MTM) leaky wave antenna (LWA) using the generated oscillating driver signal, at least one interrogating RF signal; and demodulating at least one received target reflected signal to provide thereby respective baseband signals reflected from different targets and including target-associated motion information. Different methods of sensing motion may be provided in accordance with the differing embodiments described above with respect to radio frequency (RF) motion sensors, systems, and the like.

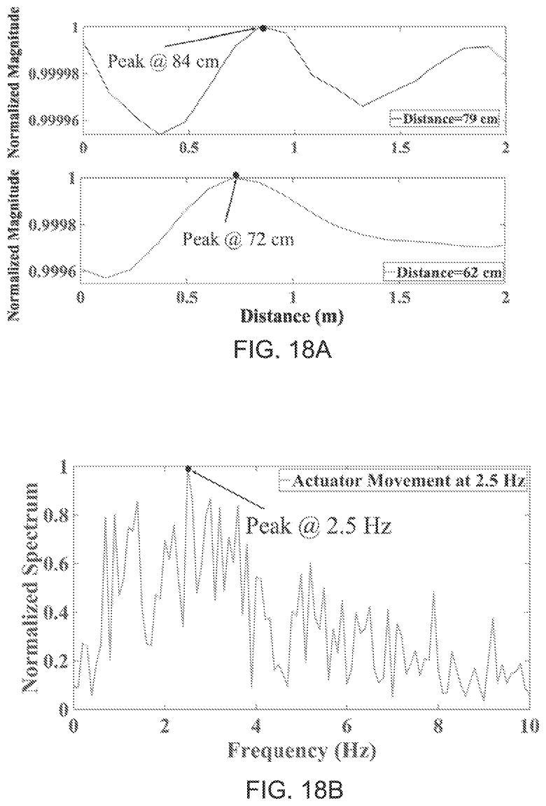

FIG. 13 depicts a high-level block diagram of a controller suitable for use in various embodiments. The controller 1305 depicted in FIG. 13 comprises a computing device that may be configured to perform various computing, processing, control, and/or other functions such as described herein with respect to the figures and equations. For example, the controller 1305 may perform various receiver or digital signal processing (DSP) functions such as described above with respect to the various figures, time domain cross-correlation functions such as described below with respect to FIG. 18, as well as various other transmitter functions, receiver functions, antenna control functions, signal generation functions, quench signal generation functions, and the like as described herein with respect to the various figures and equations. Generally speaking, in addition to performing various calculations and processing as described herein with respect to the various figures, equations, and embodiments, the controller 1305 may be used to control a motion sensing radar according to various embodiments as described herein.

As depicted in FIG. 13, the controller 1305 includes one or more processors 1310, a memory 1320, a communications interface 1330, and input-output (I/O) interface(s) 1340. The processor(s) 1310 are coupled to each of memory 1320, communication interfaces 1330, and I/O interfaces 1340.

The processor(s) 1310 are configured for controlling the operation of controller 1305, including operations supporting the methodologies described herein with respect to the various embodiments. Similarly, the memory 1320 is configured for storing information suitable for use by the processor(s) 1310. Specifically, memory 1320 may store programs 1321, data 1322 and so on. Within the context of the various embodiments, the programs 1321 and data 1322 may vary depending upon the specific functions implemented by the controller 1305. For example, as depicted in FIG. 13, the programs portion 1321 of memory 1320 includes a target detection module 1321-TD, a transmit/receive processing module 1321-TRP, a digital signal processing (DSP)/control functions module 1321-DSCP (optionally other functional elements/modules) configured to implement various computing, control, management, and/or other functions discussed in this specification.

Generally speaking, the memory 1320 may store any information suitable for use by the controller 1305 in implementing one or more of the various methodologies or mechanisms described herein. It will be noted that while various functions are associated with specific programs or databases, there is no requirement that such functions be associated in the specific manner. Thus, any implementations achieving the functions of the various embodiments may be used.

The communications interfaces 1330 may include one or more services signaling interfaces adapted to facilitate the transfer of information, files, data, messages, requests and the like between various entities in accordance with the embodiments discussed herein.

The I/O interface 1340 may be coupled to one or more presentation devices (not shown) such as associated with display devices for presenting information to a user, one or more input devices (not shown) such as touch screen or keypad input devices for enabling user input, and/or interfaces enabling communication between the controller 1305 and other computing, networking, presentation or other local or remote input/output devices (not shown).

Various embodiments are implemented using a controller 1305 comprising processing resources (e.g., one or more servers, processors and/or virtualized processing elements or compute resources) and non-transitory memory resources (e.g., one or more storage devices, memories and/or virtualized memory elements or storage resources), wherein the processing resources are configured to execute software instructions stored in the non-transitory memory resources to implement thereby the various methods and processes described herein. As such, the various functions depicted and described herein may be implemented at the elements or portions thereof as hardware or a combination of software and hardware, such as by using a general-purpose computer, one or more application specific integrated circuits (ASIC), or any other hardware equivalents or combinations thereof. In various embodiments, computer instructions associated with a function of an element or portion thereof are loaded into a respective memory and executed by a respective processor to implement the respective functions as discussed herein. Thus, various functions, elements and/or modules described herein, or portions thereof, may be implemented as a computer program product wherein computer instructions, when processed by a computing device, adapt the operation of the computing device such that the methods or techniques described herein are invoked or otherwise provided. Instructions for invoking the inventive methods may be stored in tangible and non-transitory computer readable medium such as fixed or removable media or memory or stored within a memory within a computing device operating according to the instructions.

It is contemplated that some of the steps discussed herein as software methods may be implemented within special-purpose hardware, for example, as circuitry that cooperates with the processor to perform various method steps.

Although primarily depicted and described as having specific types and arrangements of components, it will be appreciated that any other suitable types and/or arrangements of components may be used for controller 1305.

The above-described embodiments are primarily directed to SRO-based radar sensors configured to detect Doppler signatures associated with motion of multiple targets, such as vital signs (breathing and/or heartbeat) of multiple human targets. These embodiments use an amplitude modulation (AM) characteristic for SRO radar detection which is not ideal for determining target distance or ranging information.

### SRO-Based MTM Pulsed Radar Embodiments

Further embodiments are directed toward a super-regenerative oscillator (SRO)-based metamaterial (MTM) pulsed radar configured to detect multi-target vital sign signals and locations (ranging information) simultaneously. In these embodiments, a triangular waveform of 100 Hz is imposed as the quench signal to operate the radar system under the SRO mode. A MTM leaky wave antenna (LWA) is incorporated to scan from −30° to +25° with the frequency changing from 2.22 GHz to 2.79 GHz. Modulated by the quench signal, the SRO radar can generate pulse train-like waveforms. As such, the distance can be obtained by calculating the time of flight between the transmitted and received signals through the cross-correlation method in the time domain. Experimental results show that the proposed SRO-based MTM pulsed radar sensor can detect the target location accurately. Furthermore, the proposed SRO-based MTM pulsed radar can also detect Doppler signatures at the same time, where measured actuator vibration frequency as well as multi-target vital sign information agree well with the ground truth.

FIG. 14 depicts a block diagram of SRO-integrated metamaterial (MTM) leaky wave antenna (LWA) radar system 1400 for multi-target vital sign detection and location tracking in accordance with various embodiments. Specifically, FIG. 14 depicts a SRO-based MTM pulsed radar sensor 1400 similar to the integrated sensor structure 100 described above with respect to FIG. 1 that performs several functions; namely, it transmits interrogating RF signals toward each of multiple targets and receives the reflected signal(s) from the target(s). The operating frequency of the sensor system is provided by a tunable voltage-controlled oscillator (VCO) that may be used to control the illuminating direction of the MTM LWA.

The SRO-integrated sensor structure 1400 of FIG. 14 is formed using an amplifier and frequency-selective network as depicted in FIG. 1, wherein the amplifier drives the frequency-selective antenna to cause the antenna to transmit a radio frequency (RF) signal vₜᵣ (t) towards at least one target and to receive from the each target a respective reflected signal vᵣₑ(t−τₚₐₜₕ), which is detected and demodulated to produce an output signal vₒᵤₜ (t), which is fed back to the amplifier and also subjected to further processing to extract therefrom the target information such as via a signal receiver or digital signal processor (DSP) 1440, which may comprise a signal processing system having a built in analog to digital converter (ADC) or other suitable receiving/processing device. The signal receiver 1440 and a quench signal generator 1410 may be implemented in a standard manner such as by using off-the-shelf commercial equipment/components as will be appreciated by those skilled in the art, such as described below with respect to FIG. 13.

To enable the SRO-based MTM pulsed radar sensor 1400 to operate in SRO mode, a triangular (or other shape) waveform is utilized to serve as the quench signal in the oscillator circuit, which will facilitate the process of time domain cross-correlation method. The reflected signal influenced by the physiological activity of human target, such as heart shrinking and swelling, is sent into an envelope detector through an MTM-based coupler for amplitude demodulation.

To enable the SRO-based MTM pulsed radar sensor 1400 to obtain range information, RF pulses are included within each quench cycle, but with different oscillation time width for the logarithmic mode. Specifically, the receiver 1440 is configured to perform a time domain cross-correlation processing of received baseband signals to determine the time difference (Δt) between respective received and transmitted baseband signals, such as described below, which processing enables the determination of range.

In various embodiments, the quench signal is modulated according to a sequence of pulses separated by pulse intervals of a duration sufficient to allow for return of a pulse-bearing RF interrogation signal to be reflected from a target and returned to the sensor 1400. The waveshape and pulse duration of the pulse may be selected in accordance with these and other waveshapes: sinusoidal, sawtooth, triangular, etc . . . .

As shown in FIG. 14, an MTM LWA is used as a transmitting antenna (TX) to interrogate multiple targets at different locations. Signals reflected from the multiple targets are captured by a receiving antenna (RX), which is placed in proximity to the TX. The received signals are then sent through a low noise amplifier (LNA) followed by an envelope detector for data processing to obtain the range and Doppler information associated with the targets. The LNA is useful since, due to free space path loss, the amplitude of received baseband signal is much smaller than that of transmitted baseband signal.

Design of SRO-based MTM Pulsed Radar. To achieve multi-target detection, and as discussed in more detail above, the transmitter consists of a voltage-controlled oscillator (VCO) and a one-dimensional (1D) MTM LWA. The oscillator design is based on a negative resistance method with a common source configuration in which a quench signal is imposed to enable the SRO mode operation, while the MTM LWA is integrated with an MTM-based coupler such that a portion of the transmitted signal can be directly coupled into an envelope detector.

In an experimental configuration, both MTM LWA and oscillator are fabricated using Rogers RT/duroid 5880 with a thickness of 1.27 mm, a dielectric constant of 2, and a loss tangent (tan δ) of 0.0027. Further, a Vivaldi antenna is employed to receive the modulated signal reflected from the target(s), and a LNA followed by another envelope detector is used to demodulate the amplitude variation. It is noted that a metal plate is placed between the TX and RX to mitigate the TX leakage.

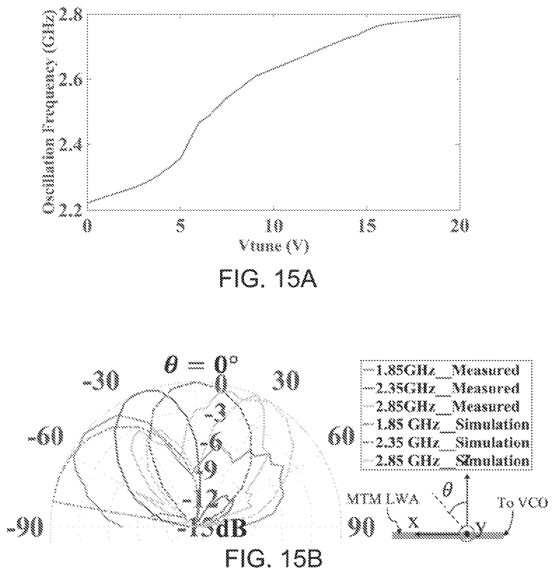

FIG. 15A graphically illustrates measured oscillation frequency as a function of varactor bias voltage (Vₜᵤₙₑ) of a 1D MTM LWA in the system of FIG. 14. It can be seen that the oscillation frequency of the VCO in this embodiment may be tuned from 2.22 GHz to 2.79 GHz when the bias voltage of varactor is swept from 0 to 20 V.

FIG. 15B graphically illustrates a radiation pattern of a 1D MTM LWA in the system of FIG. 14 according to an embodiment. It can be seen that the radiation angle of the main beam can be scanned from −30° to +25° with the oscillation frequency varying from 2.22 GHz to 2.79 GHz. Multiple targets can thus be detected from the backward to forward region using the SRO MTM pulsed radar sensor of the various embodiments.

To detect the vital sign information and location simultaneously, the proposed SRO-based MTM pulsed radar sensor of FIG. 14 is operated in the logarithmic mode, where a triangular waveform serving as a quench signal is imposed at the drain of transistor for the VCO.

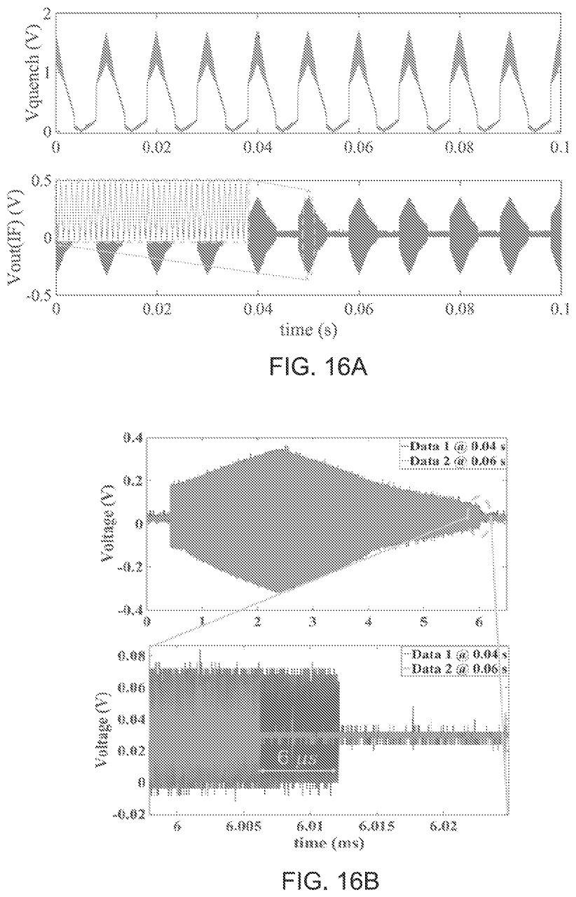

FIG. 16A graphically illustrates a measured time-domain waveform of an output signal from an MTM-based coupler (down converted to 3 MHz and having a quench signal at 100 Hz). An oscilloscope with a sampling rate of 100 MHz is used to capture the output signal from MTM-based coupler and the quench signal. Specifically, FIG. 16A illustrates a time domain quench waveform (V_{quench}) at 100 Hz and the down-converted output signal (V_{out(IF)}) at 3 MHz. Due to the parasitic capacitor of oscillator circuit, a small waveform distortion of quench signal can be observed.

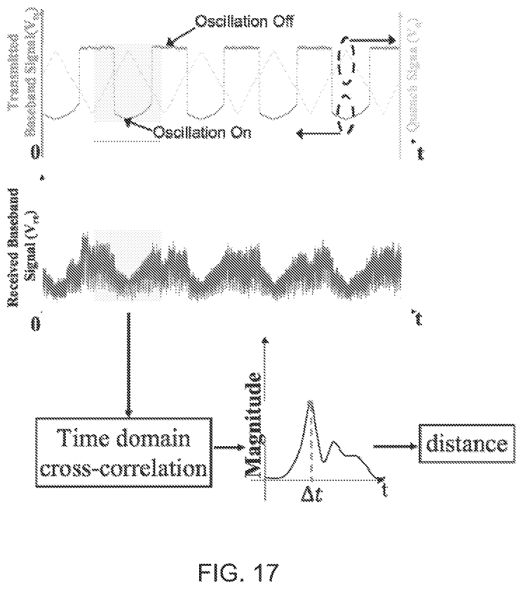

FIG. 16B graphically illustrates a comparison of oscillation time width between different quench cycles (without an envelope detector). Specifically, FIG. 16B illustrates the output signal of SRO-based MTM LWA is composed of RF pulses within each quench cycle, but with different oscillation time width for the logarithmic mode. Moreover, due to free space path loss, the amplitude of received baseband signal is much smaller than that of transmitted baseband signal. To this end, a time domain cross-correlation method may be employed to extract the time difference (Δt) between the received and transmitted baseband signals, as shown in FIG. 17.

FIG. 17 graphically depicts distance measurement using a cross-correlation method in the time domain according to an embodiment. In particular, a distance (D) between the radar sensor and the target can be calculated as follows:

$$D = \frac{c \cdot Δ t}{2} \qquad ( 10 )$$

where c is the speed of light.

Combining with the angular information provided by the radiation pattern of MTM LWA, the location of target can be obtained, i.e., the angular direction can be obtained from the frequency-space mapping relation of MTM LWA and the distance can be obtained from (10).

To demonstrate the feasibility of the designed SRO-based pulsed radar sensor, an actuator with a displacement of 5 mm and the vibration frequency of 2.5 Hz is utilized.

In one experiment, a triangular waveform at the frequency of, illustratively, 100 Hz with a 10 ms or shorter pulse width and an amplitude of 2.5 V and a dc offset of 1.25 V is imposed as the quench signal. An actuator is placed in front of the SRO radar system at distances of 62 cm and 79 cm, respectively. A Tektronix MDO34 oscilloscope with a sampling rate of 1.25 GHz is used to acquire the baseband signal from the transmitter and the receiver side simultaneously. In this configuration, the minimal detectable time difference, which is limited by the oscilloscope's sampling rate, is 0.8 ns, thereby resulting in a round-trip distance resolution of 24 cm. It is noted that other pulse waveshapes, pulse widths, frequencies (e.g., 50 Hz to 1000 Hz, greater than 1000 Hz, etc.) and the like may also be used within the context of the various embodiments.

By using the time domain cross-correlation method, a strong correlation peak between the transmitted and received baseband signals can be found at t=Δt. Then the distance between the actuator and radar sensor can be calculated based on (10), which is shown in FIG. 18A, which graphically illustrates measured distance results associated with an experiment using an actuator at distances of 79 cm and 62 cm. A small deviation between the measured results and ground truth may be due to the distance resolution limitation. In addition, the lower peaks at 0 m and 1.8 m are mainly caused by the reflected signals from adjacent objects, such as walls. As such, in practice, without the presence of the abosorbers, multiple peak values may be observed by using the time-domain cross-correlation method, due to unexpected reflected signals from adjacent objects. In this scenario, additional data-post processing methods, such as digital range filter window, can be incorporated to obtain the desired actual distance for the target.

Furthermore, by performing fast Fourier Transform (FFT) on the received baseband signal, the vibration frequency of actuator can be obtained in the frequency spectrum, as shown in FIG. 18B, which graphically received spectrum associated with the experiment using an actuator at 62 cm an agrees well with the ground truth.

The various embodiments provide a system capable of multiple target vital sign and location detection. Specifically, the various embodiments provide a SRO-based MTM pulsed radar sensor that is also used to detect vital signs and locations for multiple targets.

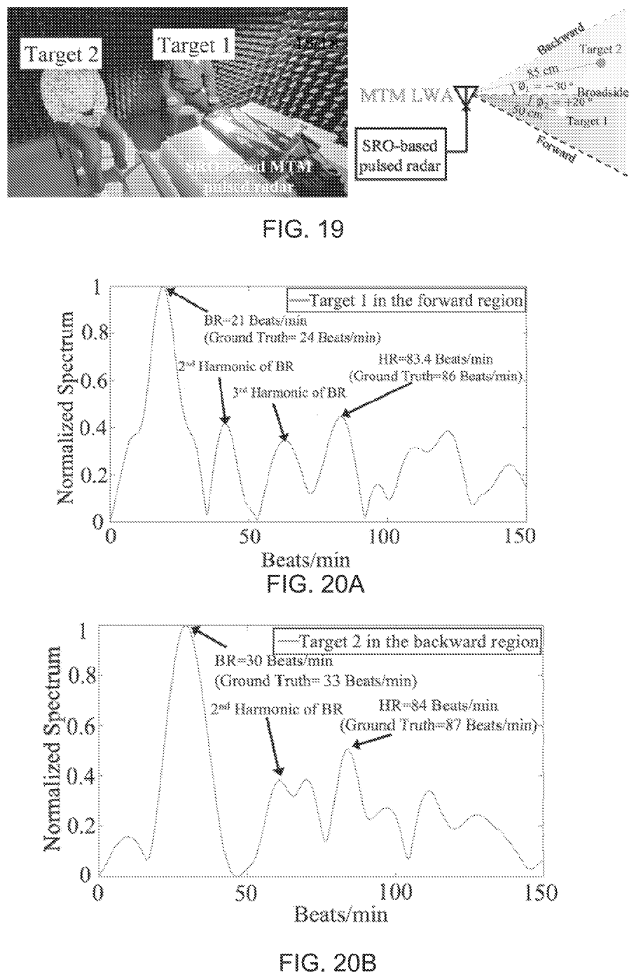

FIG. 19 depicts an experiment setup and top-view sketch of a SRO-based MTM pulsed radar for multi-target vital sign detection and location tracking. It can be seen that Target 1 is along the direction of Ø=+20° with a distance of 0.5 m in the forward region. Based on the radiation pattern in FIG. 15B, the corresponding frequency provided by oscillator is 2.68 GHz. Target 2 is along the direction of Ø=−30° with a distance of 0.85 m in the backward region, in which the corresponding frequency provided by oscillator is 2.3 GHz. A smart watch is used to measure the ground truth of HR, while the ground truth of BR is counted by the targets themselves. By performing FFT on the received baseband signal, the BR and HR for both targets can be extracted in the frequency spectrum, which are plotted in FIGS. 20A-20B, graphically illustrates measured breathing rate and heartbeat for the first and second targets, respectively, in the experiment.

Furthermore, the distance information of the targets can be calculated through the use of the cross-correlation method in the time domain, in which the time difference between the transmit and receive signals can be obtained.

TABLE 1

| | Target 1 | Target 2 |
|---|---|---|
| Distance (m) | 0.6 | 0.72 |
| Ground truth (m) | 0.5 | 0.85 |
| BR (beats/min) | 21 | 30 |
| Ground Truth (beats/min) | 24 | 33 |
| HR (beats/min) | 83.4 | 84 |
| Ground Truth (beats/min) | 86 | 87 |

Table 1 summarizes the measured vital sign information and distances for both targets at different locations. It can be observed that the measured BR and HR are close to the ground truth with a small deviation, which might be caused by the measurement errors between the smart watch (utilized here to obtain the ground truth) and the proposed SRO pulsed radar, whereas a small difference in range detection between the measured results and ground truth is mainly caused by the distance resolution limitation from the oscilloscope. To improve the distance resolution, a high-speed data acquisition device can be adopted to achieve a smaller detectable time difference.

As discussed above, a new kind of SRO-based MTM pulsed radar is disclosed herein that is suitable for use to detect multi-target vital sign information and locations simultaneously. A 1D MTM LWA is used in the SRO-based pulsed radar to scan from the backward the forward directions, where the distance between the radar system and target can be calculated based on the time difference between the transmitted and received pulse waveforms by using the time domain cross-correlation method. Meanwhile, the vital sign information can be extracted by performing FFT on the received baseband signals. As such, both the distance and vital sign information of targets at different locations can be obtained accurately through the disclosed SRO-based MTM pulsed radar system.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A radio frequency (RF) motion sensor, comprising:
a metamaterial (MTM) leaky wave antenna (LWA) configured to transmit an interrogating RF signal toward each of a plurality of targets and to correspondingly receive therefrom respective target reflected signal;
a super-regenerative oscillator (SRO) comprising a tunable voltage-controlled oscillator (VCO) configured to generate for transmission by the MTM LWA an oscillating driver signal in accordance with a sequence of quench cycles, wherein an amplitude of the oscillating signal changes from an initial value to an equilibrium value (V_{eq}) and back to an extinction value during a first portion of each quench cycle; and a demodulator configured to demodulate from the MTM LWA received signals to provide thereby respective baseband signals reflected from different targets, which includes the target-associated motion information.

2. The RF motion sensor of claim 1, wherein the MTM LWA comprises a one-dimensional (1D) MTM LWA.

3. The RF motion sensor of claim 1, wherein the MTM LWA comprises a two-dimensional (2D) MTM LWA.

4. The RF motion sensor of claim 1, wherein the VCO has an oscillation band of between approximately 2 GHz and 2.5 GHz.

5. The RF motion sensor of claim 1, wherein a tuning voltage Vₜᵤₙₑ for controlling the output oscillation frequency of the VCO is generated using a function generator.

6. The RF motion sensor of claim 1, wherein a tuning voltage Vₜᵤₙₑ for controlling the output oscillation frequency of the VCO is generated using a DC power supply.

7. The RF motion sensor of claim 1, wherein MTM LWA generates a main beam having a direction θ(ω) expressed as:
$$θ ( ω ) = arc sin ( \frac{β ( ω )}{{k}_{0}} ) .$$

8. The RF motion sensor of claim 1, wherein the amplitude of the oscillating driver signal changing during the first portion of each quench cycle exhibits an exponential function in amplitude.

9. The RF motion sensor of claim 8, wherein the exponential function in amplitude is expressed as:
$${V}_{e q k} = {V}_{rek} {e}^{\frac{{t}_{k}}{{τ}_{0 k}}} .$$

10. The RF motion sensor of claim 1, wherein each target comprises a human and the target reflected signal is configured to enable detection of cardiac activity of each human.

11. The RF motion sensor of claim 1, wherein the quench signal is modulated according to a sequence of pulses separated by a pulse interval such that each target reflected signal includes respective target ranging information.

12. The RF motion sensor of claim 11, wherein target ranging information is retrieved from each target reflected signal using time domain cross-correlation.

13. The RF motion sensor of claim 11, wherein each of the sequence of pulses comprises a 100 Hz signal having predefined pulse width and pulse interval of less than 10 ms, the signal comprising one of a sinusoidal, a sawtooth, and a triangular waveshape.

14. A system configured to detect cardiac activity of a plurality of humans, the system including a radio frequency (RF) motion sensor, comprising:
a metamaterial (MTM) leaky wave antenna (LWA) configured to transmit an interrogating RF signal toward each of a plurality of human targets and to correspondingly receive therefrom respective target reflected signal;
a super-regenerative oscillator (SRO) comprising a tunable voltage-controlled oscillator (VCO) configured to generate for transmission by the MTM LWA an oscillating driver signal in accordance with a sequence of quench cycles, wherein an amplitude of the oscillating signal changes from an initial value to an equilibrium value (V_{eq}) and back to an extinction value during a first portion of each quench cycle; and a demodulator configured to demodulate from the MTM LWA received signals to provide thereby respective baseband signals reflected from different targets, which includes the target-associated motion information.

15. The system of claim 14, wherein the MTM LWA comprises a two-dimensional (2D) MTM LWA, and system is configured to perform frequency-dependent space mapping.

16. The system of claim 14, wherein each target comprises a human and the target reflected signal is configured to enable detection of cardiac activity of each human.

17. The system of claim 14, wherein the quench signal is modulated according to a sequence of pulses separated by a pulse interval such that each target reflected signal includes respective target ranging information.

18. The system of claim 17, wherein target ranging information is retrieved from each target reflected signal using time domain cross-correlation.

19. A method of sensing motion, comprising:
generating, using a super-regenerative oscillator (SRO) comprising a tunable voltage-controlled oscillator (VCO), an oscillating driver signal in accordance with a sequence of quench cycles, wherein an amplitude of the oscillating signal changes from an initial value to an equilibrium value (V_{eq}) and back to an extinction value during a first portion of each quench cycle;
transmitting, via a metamaterial (MTM) leaky wave antenna (LWA) using the generated oscillating driver signal, a plurality of interrogating RF signals; and
demodulating at least two received target reflected RF signals to provide thereby respective baseband signals reflected from different targets and including target-associated motion information.

20. The method of claim 19, wherein:
each target comprises a human and the target reflected signal is configured to enable detection of cardiac activity of each human;
the quench signal is modulated according to a sequence of pulses separated by a pulse interval such that each target reflected signal includes respective target ranging information, the target ranging information being retrieved from each target reflected signal using time domain cross-correlation.
